(19) United States
(12) Patent Application Publication
(10) Pub. No.: US 2017/0119849 A1
(43) Pub. Date: May 4, 2017

(54) Therapy of Cardiomyopathy by Intramyocardial Cell Delivery and Cytokine Administration

(71) Applicants: REGENERATE LIFE SCIENCE LIMITED, London (GB); BARTS HEALTH NHS TRUST, London (GB); QUEEN MARY UNIVERSITY OF LONDON, London (GB)

(72) Inventors: Anthony MATHUR, London (GB); John Francis MARTIN, London (GB)

(21) Appl. No.: 15/317,833

(22) PCT Filed: Jun. 10, 2015
(86) PCT No.: PCT/GB2015/051714

§ 371 Date: Dec. 9, 2016

(30) Foreign Application Priority Data
Jun. 10, 2014 (GB) 1410334.5

(51) Int. Cl. A61K 38/19; A61K 9/00; A61B 5/00; A61K 35/28

(52) U.S. Cl. CPC A61K 38/193; A61K 35/28; A61K 9/0019; A61B 5/4887; A61K 2035/124

(57) ABSTRACT

The present invention provides a composition comprising cells obtainable by bone marrow aspiration, or multipotent or pluripotent progenitor cells, and granulocyte-colony stimulating factor (G-CSF), as a combined preparation for simultaneous, separate or sequential use in the therapy of cardiomyopathy in a subject, wherein the cells are administered directly to the myocardium of the subject.

### FIELD OF THE INVENTION

The present invention concerns the treatment of cardiomyopathy, especially by intramyocardial cell delivery and adjunct cytokine administration.

### BACKGROUND TO THE INVENTION

One of the principal causes of heart failure in Western countries is ischaemic heart disease. Despite advances in treatment, the prognosis for patients who are admitted to hospital with heart failure remains poor, with a 5-year survival of approximately 50% and a 10 year survival of around 10% (Macintyre et al., 2000; Mosterd et al., 2001). The increasing prevalence of heart failure poses a significant burden to patients, practitioners, and healthcare systems and hence defines a need for new treatments.

Granulocyte-colony stimulating factor (G-CSF) is a potent cytokine often used for the purpose of pre-mobilisation of progenitor cells. A few trials have also looked at whether treatment with G-CSF alone leads to improvement in cardiac function. Some of the results have suggested an improvement in cardiac function with G-CSF although most trials have been small and the overall results have been mixed (Hill et al., 2005; Wang et al., 2005; Honold et al., 2012; Joseph et al., 2005). Phase I/II trials using autologous progenitor cells in ischaemic cardiomyopathy have shown promising results although these studies have often lacked an appropriate comparison group for the intervention. Only a small number of trials have assessed the use of cytokine therapy as an adjunct to cell therapy (Jeevanantham et al., 2012; Fisher et al., 2014).

The present invention concerns the outcome of the phase II clinical trial REGENERATE-IHD. The study design, but not results, of the randomised, placebo-controlled trial, conducted in part by the present inventors, is known to the skilled person and is described in Yeo & Mathur (2009), and available at clinicaltrials.gov under the registration number NCT00747708 (https://clinicaltrials.gov/ct2/show/NCT00747708?term=NCT00747708&rank=1, as accessed on 10 Jun. 2014). The study sought to address for the first time whether cytokine therapy using G-CSF alone, or when combined with cell therapy (administration of autologous bone marrow-derived cells), has an ameliorative effect on patients with ischaemic cardiomyopathy. The trial also sought to compare the intracoronary and intramyocardial delivery routes for the autologous bone marrow-derived cells, with regard to both safety and efficacy.

A number of hypotheses were made at the outset of the trial. Notably, that both the intracoronary and intramyocardial injection routes for autologous cells would both confer an improvement in cardiac function and symptoms beyond that of G-CSF administration alone. It was also hypothesised that G-CSF administration, in the absence of cell therapy, would confer some improvement in cardiac function and symptoms, resulting from an increase in the circulation of CD34+ progenitor cells. The trial was begun in August 2005, with the final data collection date in May 2012.

Running parallel to REGNERATE-IHD was the FOCUS-CCTRN phase II clinical trial (Perin et al., 2012), from March 2009 to November 2011. The study design and results of the trial are known to the skilled person, and available at clinicaltrials.gov under the registration number NCT00824005 (https://clinicaltrials.gov/ct2/show/study/NCT00824005?term=NCT00824005&rank=1, as accessed on 10 Jun. 2014), in addition to the above publication. The trial sought to establish whether autologous bone marrow-derived cells delivered by the intramyocardial route would improve myocardial perfusion, left ventricular end-systolic volume or maximal oxygen consumption in patients with coronary artery disease or left ventricle dysfunction, and limiting heart failure or angina. No significant improvements in primary or secondary endpoints were observed by the authors.

Thus, there remains a need in the art for an effective therapy for the treatment of disorders such as ischaemic cardiomyopathy.

### SUMMARY OF THE INVENTION

The present invention is based at least in part on the REGENERATE-IHD randomised trial of adjunct cytokine therapy and autologous bone marrow-derived cells transplanted to the myocardium of patients with ischaemic cardiomyopathy. The trial achieved its primary end-point by demonstrating a 4.99 percentage point increase in left ventricular ejection fraction (LVEF) within the intramyocardial (IM) route cell-treated plus adjunct G-CSF group at 1 year. However, contrary to the initial hypotheses of the trial, this degree of improvement in LVEF was only seen in the IM cell-treated plus adjunct G-CSF group and, importantly, not in any other group (G-CSF administration alone, or G-CSF in conjunction with cell therapy administered by the intracoronary (IC) route). This also stands in contrast to the FOCUS-CCTRN trial, in which patients receiving autologous cell therapy by the IM route showed no improvement in the primary end-points of the trial.

The protocol of REGNERATE IHD differed from that of FOCUS-CCTRN by the administration of, additionally, G-CSF and not IM-delivered cell therapy alone, but also in a number of parameters concerning the preparation and delivery of the cells. Said parameters are not described in the study design published by Yeo & Mathur (2009) and are not disclosed in the study methodology available on clinical trials.gov (NCT00747708). They are defined for the first time in the Detailed Description of the Invention (below). The combination of G-CSF administration with cell therapy in accordance with said parameters is shown to result in a significant improvement in cardiac function, as measured primarily by LVEF. This effect is not seen in the FOCUS-CCTRN trial, the methodology of which lacks said features.

According to a first aspect of the invention, there is provided a composition comprising cells obtainable by bone marrow aspiration and granulocyte-colony stimulating factor (G-CSF), as a combined preparation for simultaneous, separate or sequential use in the therapy of cardiomyopathy in a subject, wherein the cells are administered directly to the myocardium of the subject. Preferably, the G-CSF is administered subcutaneously.

According to a second aspect of the invention, there is provided a composition comprising multipotent or pluripotent progenitor cells and granulocyte-colony stimulating factor (G-CSF), as a combined preparation for simultaneous, separate or sequential use in the therapy of cardiomyopathy in a subject, wherein the cells are administered directly to the myocardium of the subject. Preferably, the G-CSF is administered subcutaneously.

Further aspects are defined in the claims.

### BRIEF DESCRIPTION OF THE FIGURES

FIG. 1. Flow chart of the study summarising the study design and number of participants at each stage of the trial.

FIG. 2. Primary endpoint analysis of left ventricular ejection fraction. Results of change in LVEF at one year from baseline as measured using CMR/CT in each of the treatment groups (A-F). A-peripheral placebo; B—peripheral G-CSF only; C—peripheral G-CSF+intracoronary serum; D—peripheral G-CSF+intracoronary bone marrow-derived cells (BMC); E—peripheral G-CSF+intramyocardial serum; F—peripheral G-CSF+intramyocardial BMC. A significant improvement of 4.99 percentage point was observed in the intramyocardial BMC treated group (group F). * denotes p<0.05. Large solid circles represent the means at baseline and 1 year for each group respectively, and error bars represent 95% Cls.

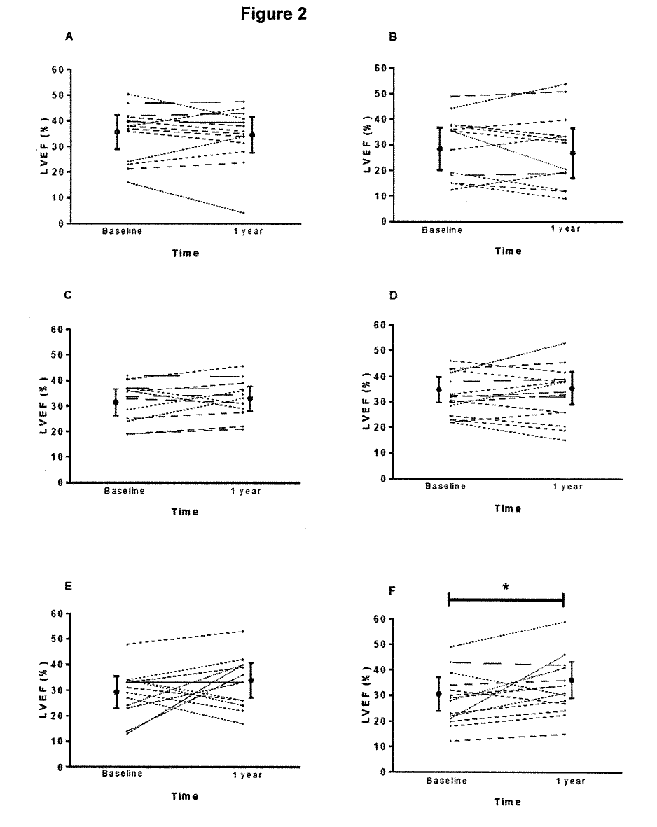

FIG. 3. Change in NT pro-BNP. Change in NT pro-BNP at 6 months compared to baseline is depicted using box and whisker plots (median and range) on a logarithmic scale. NT pro-BNP was significantly reduced in the intramyocardial (IM) BMC group. A—peripheral placebo; B—peripheral G-CSF only; C—peripheral G-CSF+intracoronary serum; D—peripheral G-CSF+intracoronary bone marrow derived cells (BMC); E—peripheral G-CSF+intramyocardial serum; F—peripheral G-CSF+intramyocardial BMC; * denotes p<0.05

FIG. 4. Change in NYHA. Mean NYHA class at 1 year as compared to baseline is shown for each of the treatment groups (A-F, as described above). A significant reduction in NYHA class was noted in the intramyocardial bone marrow cell (BMC) group. * denotes p<0.05; Significance assessed using one-way ANOVA with Bonferroni correction for multiple comparisons.

FIG. 5. Schematic of a bone marrow sample (A) before and (B) after centrifugation during manual bone marrow aspirate processing.

### DETAILED DESCRIPTION OF THE INVENTION

As described above, the REGENERATE-IHD trial of combined cytokine therapy and autologous bone marrow-derived cells transplanted to the myocardium of patients with ischaemic cardiomyopathy achieved its primary end-point by demonstrating a 4.99 percentage point increase in left ventricular ejection fraction (LVEF) within the IM route cell-treated plus subcutaneous G-CSF group at 1 year. The achievement of the primary end-point in the IM-delivered plus subcutaneous G-CSF group is also supported by improvements in the clinical endpoint of New York Heart Association (NYHA) functional class and a fall in the biochemical marker N-terminal of the prohormone brain natriuretic peptide (NT pro-BNP). Cell function as measured by CFU assay was directly correlated to change in LVEF seen in the IM cell group supporting the hypothesis that the cells themselves are mechanistically implicated in the sustained improvement in cardiac function. The present inventors' findings confirm that G-CSF alone has no beneficial effect on patients with advanced heart failure but that the combination with intramyocardial stem cell injection appears to improve clinical and functional parameters.

G-CSF, aside from being advocated as an adjunct for cell mobilisation, has been postulated as an independent therapeutic agent in ischaemic cardiomyopathy with small previous studies suggesting an associated improvement in LVEF (Joseph et al., 2006). The REGENERATE-IHD trial is the first, randomised, controlled trial to demonstrate that isolated G-CSF treatment has no beneficial effect on functional, clinical and biochemical markers of heart failure in this patient population. Previous positive studies using G-CSF have either lacked appropriate controls or have not been randomised controlled studies (Hill et al., 2005; Wang et al., 2005; Honold et al., 2012; Joseph et al., 2005; Chih et al., 2012).

In REGENERATE-IHD, all interventional arms received G-CSF, but only patients receiving G-CSF and intramyocardial cell injection showed a benefit. This is in contrast to the FOCUS-CCTRN trial in which patients receiving autologous cell therapy by the IM route showed no improvement in the primary end-points of the trial. Although not directly tested here due to differences in other parameters (below), this may suggest that G-CSF is needed in addition to IM cell injection to demonstrate benefit in heart failure patients

In addition to the improvement in myocardial function, the present inventors observed improvements in both functional and biochemical parameters in the IM cell-treated plus subcutaneous G-CSF group. Firstly, a sustained reduction in NYHA functional class at 1 year, associated with quality of life (QoL) improvement, suggests an improvement in exercise capacity in this group. Additionally in this same group, a significant improvement in NT pro-BNP was noted supporting this improved LVEF. Although significant improvement NYHA functional class, a trend towards significant reduction in NT pro-BNP and improvements in certain QoL parameters were seen in the IC cell-treated group, the lack of improvement in objective cardiac functional parameters makes a clinical benefit less likely.

The baseline characteristics and LVEF (<30%) were consistent with a patient population with advanced ischaemic cardiomyopathy. These were well matched across the groups. No difference in the functional capacity (as measured by colony forming units (CFU) of cells between the groups was seen following 5 days of G-CSF therapy. Furthermore, positive evidence of a correlation between CFU and ejection fraction in the IM cell-treated patients has not previously been observed in a randomised trial. This for the first time supports a causal relationship between cell function and cardiac function. An inverse association has previously been demonstrated between CFU numbers and mortality in a registry study (Assmus et al., 2007).

The use of autologous cells in patients with advanced heart failure appears safe with very low peri-procedural complication rates. Furthermore, the rise in troponin in the IM group is in keeping with previously published literature (Baldazzi et al., 2008), and interestingly appears localised to the placebo arm. Both interventions i.e. IM and IC injection of cells appear to be safe in terms of low long term risk of significant arrhythmias, low rate of admissions with exacerbation of heart failure and low major adverse cardiac event (MACE) rates (only 2 events in the whole trial at 1 year). Although this trial was not powered to detect significant differences in MACE or mortality the data is in keeping with other similar trials and reconfirms the safety and feasibility of using autologous bone marrow derived cells in chronic heart failure patients.

With regard to the procedure of the trial, the following features differentiate REGENERATE-IHD from FOCUS-CCTRN, in which no improvements in cardiac function were observed as a result of IM autologous bone marrow-derived cell therapy. Firstly, cytokine therapy by pre-treatment with G-CSF (for 5 days by subcutaneous injection, prior to IM cell delivery) was undertaken, a feature absent from FOCUS-CCTRN. Secondly, areas of myocardium were selected by NOGA® electromechanical mapping having a unipolar voltage between 6.9 mV and 11 mV, as sites of injection. Values below 6.9 mV are characteristic of scar tissue, and those above 11 mV indicate the area of the myocardium to be overactive; in FOCUS-CCTRN only the minimum value of 6.9 mV was considered, without any upper limit for the selection of injectable sites (Perin et al., 2012). In addition to the above, in FOCUS-CCTRN the bone marrow aspirate, once isolated, was processed by a closed automated cell processing system (Sepax, Biosafe SA) (Perin et al., 2012). In contrast, in REGENERATE-IHD, a manual cell processing protocol was followed (see Standard Operating Procedure, Example), the essential features of which are defined below. As no single variable, out of the above, is considered in isolation in comparison to FOCUS-CCTRN, it is concluded by the present inventors that a composite of the above features of the trial methodology enabled the achievement of the primary end-point.

As used herein, “therapy” comprises both “treatment” and “prevention”.

As used herein, the terms “treating” and “treatment” and “to treat” refer to therapeutic measures that cure, slow down, and/or halt progression of a diagnosed pathologic condition or disorder. Thus, those in need of treatment include those already having the disorder. As particularly, but not necessarily, envisaged by the present inventors, a subject is successfully “treated” for a cardiomyopathy according to the present invention upon achievement of the primary end-point of the REGENERATE-IHD trial, when following analogous or identical protocols over a comparable or identical timescale. The primary end-point for all patients was the significant change in left ventricular ejection fraction (LVEF) at 12 months compared to baseline as measured using advanced cardiac imaging (cardiac magnetic resonance imaging (CMR) or computed tomography (CT) where CMR contraindicated). A subject may also be considered successfully “treated” upon achieving one or more secondary end-points of the REGENERATE-IHD trial, preferably when in combination with achievement of the primary end-point, when following analogous or identical protocols over a comparable or identical timescale. Secondary endpoints included change in NT-pro BNP, NYHA, quality of life (assessed by EQ5D®, SF-36® and MacNew® questionnaires) and LVEF assessed using contrast transthoracic echocardiogram (TTE) and Quantitative Left Ventriculography (QLV) at 6 months compared to baseline. Additional secondary endpoints at 1 year were change in NYHA class, LVEF measured by contrast TTE and quality of life compared to baseline.

As used herein, the term “cardiomyopathy” has its conventional meaning as used in the art; that is, generally, the deterioration of the function of the myocardium (the muscle of the heart) for any reason. “Ischaemic cardiomyopathy” refers to a weakness in the muscle of the heart due to inadequate oxygen delivery to the myocardium, generally due to the absence or relative deficiency of its blood supply.

As used herein, the term “myocardium” has its conventional meaning as used in the art; that is, generally, the muscle of the heart. Direct administration of a composition comprising cells obtainable by bone marrow aspiration to the myocardium of the subject is envisaged by the present invention, meaning the composition is transferred from the device of administration (particularly envisaged, an injection catheter) to the myocardium tissue without having traversed any intervening tissue (for example coronary blood vessels). “Intramyocardial injection” refers to direct administration to the myocardium of the subject by injection.

As used herein, the term “bone marrow” has its conventional meaning as used in the art; that is, generally, the gelatinous tissue present in bone cavities. The tissue comprises red bone marrow, a subset of bone marrow having populations of inter alia, adult stem cells, progenitor cells and precursor cells contained therein. The term “bone marrow-derived”, as used herein with reference to cells (particularly autologous cells), refers to said cells having been extracted from bone marrow, in particular by aspiration of the bone marrow of the subject.

As used herein, the term “bone marrow aspiration” has its conventional meaning as used in the art; that is, generally, the removal of a quantity of bone marrow (generally, but not necessarily, in liquid form). Cells may be considered “obtainable” by the procedure if reasonably expected by the skilled person to be removed from the bone marrow thereby (in other words, the cells would be reasonably expected to reside in the bone marrow, prior to aspiration, and be removable by the procedure); additionally, cells derived from such cells (for example progeny of such cells obtained by in vitro or ex vivo propagation) are within the scope of the invention, even if having greater lineage restriction (i.e. differentiation) than such (parent) cells. Thus, cells “obtainable” by bone marrow aspiration includes cells obtained by the procedure, cells which would reasonably be expected by the skilled person to be obtained by the procedure, and the progeny of either of the above (particularly resulting from in vitro or ex vivo expansion). Both autologous and allogeneic cells obtainable by bone marrow aspiration are within the scope of the invention, although autologous cells are particularly envisaged. Human embryonic stem cells or any cell resulting from the destruction of human embryos are not within the scope of the invention.

As used herein, the term “multipotent or pluripotent progenitor cell” refers to an undifferentiated cell that can be induced to proliferate, and is not restricted to a single lineage (that is, not committed to differentiate into a single cell type). As used herein, the term is interchangeable with the term “stem cell”, as used in the art. Both autologous and allogeneic mulitpotent or pluripotent progenitor cells are within the scope of the invention, although autologous cells are particularly envisaged. However, human embryonic stem cells or any cell resulting from the destruction of human embryos are not within the scope of the invention. The above cell is capable of self-maintenance, meaning that with each cell division, one daughter cell will also be a cell capable of multi-lineage differentiation. The term also encompasses progeny of the above cell, for example progeny obtained by in vitro or ex vivo propagation of the cell, for example from a sample obtained from a subject. Multipotent or pluripotent progenitor cells can be obtained from a number of tissues, however adult tissue comprising said undifferentiated cells is particularly envisaged, for example the bladder, amniotic fluid and/or bone marrow, bone marrow being an especially envisaged source. Multipotent or pluripotent progenitor cells may also be, or be derived from (i.e. be progeny of), induced pluripotent stem cells, for example those derived from adult dermal fibroblasts, or any other suitable cell type, using standard methods known in the art.

As used herein, the term “subject” refers to any animal (for example, a mammal), including, but not limited to, humans, non-human primates, canines, felines, rodents, and the like, which is to be the recipient of a therapy for cardiomyopathy in accordance with the use of the present invention. Human subjects are envisaged in particular. “Patient” is used herein to refer to a human subject.

As used herein, the term “multipotent or pluripotent adult stem cell” refers to a stem cell present in or obtained from (such as isolated from) an organism after birth, for example the subject (if said cells are allogeneic); the cell being undifferentiated, capable of being induced to proliferate, not restricted to a single lineage (that is, not committed to differentiate into a single cell type), and capable of differentiating into different cell types from the same embryonic layer. The above cell is capable of self-maintenance, meaning that with each cell division, one daughter cell will also be a multipotent or pluripotent adult stem cell. Haematopoietic stem cells, obtainable from the bone marrow of a subject, are a particularly envisaged type of multipotent or pluripotent adult stem cell for use according to the present invention.

As used herein, the term “bone marrow stem cell” refers to any adult stem cell capable of multi-lineage differentiation present in bone marrow, and particularly those which are present in or may be (at least partly) isolated from a sample of bone marrow aspirate; for example, a sample taken from the subject.

As used herein, the term “CD34+” with reference to a cell (in particular, a bone marrow stem cell), refers to the presence of the cell-cell adhesion factor Cluster of Differentiation 34 on the surface of said cell, that is generally expressed on cells associated with early haematopoietic and vascular-associated tissues. CD34+ cells are identifiable by a number of standard methods known in the art, in particular flow cytometry.

As used herein, the term “endothelial progenitor cell” refers to progenitor cells having the ability to differentiate into endothelial cells, thus being able to participate in inter alia, vasculogenesis and/or vascular homeostasis.

As used herein, the term “autologous”, with respect to a cell, has its conventional meaning as used in the art; that is, generally, the cell having been taken from the subject (for example by bone marrow aspiration) or derived from such a cell (for example, progeny of a cell taken from a patient and subsequently expanded ex vivo).

As used herein, the term “granulocyte-colony stimulating factor” (G-CSF) has its conventional meaning as used in the art, as would be understood by the skilled person. Generally, G-CSF is a cytokine having the ability to stimulate the proliferation and differentiation of haematopoietic precursor cells in, especially, the bone marrow.

As used herein, the term “percentage point” refers the absolute difference in a parameter that is measured as a percentage (for example left ventricular ejection fraction—LVEF). By way of example, a 4.99 percentage point increase from a baseline LVEF of 30% within 1 year results in a final LVEF of 34.99%, and not 31.497%.

The composition, kit and method of the invention provides for a “kit of parts”, i.e. the administration of more than one component in a simultaneous, sequential or separate manner. For the avoidance of doubt, it is not necessary that the individual components of the kit/composition are packaged together (but this is one embodiment of the invention). It is also not necessary that they are administered at the same time. As used herein, “separate” administration means that the components are administered as part of the same overall dosage regimen (which could comprise a number of days). As used herein “simultaneous” means that the components are to be taken together or formulated as a single composition. As used herein, “sequential” means that the components are administered at about the same time, and preferably within about 1 hour of each other.

According to a first aspect of the invention, there is provided a composition comprising cells obtainable by bone marrow aspiration and granulocyte-colony stimulating factor (G-CSF), as a combined preparation for simultaneous, separate or sequential use in the therapy of cardiomyopathy in a subject, wherein the cells are administered directly to the myocardium of the subject. Preferably, the G-CSF is administered subcutaneously. According to said first aspect, said cells are obtainable by bone marrow aspiration, thereby including cells obtainable by any suitable embodiment of such a procedure as described in the art. As an example only, it is possible for bone marrow to be obtained from the posterior iliac crest of the subject; between 20 and 120 ml, preferably between 30 and 100 ml, more preferably between 40 and 80 ml, most preferably about 50 ml bone marrow may be aspirated from 2, 3, 4 or more different sites over the said iliac crest. The bone marrow aspirate, if obtained correctly, will inherently comprise the desired cells.

According to a second aspect of the invention, there is provided a composition comprising multipotent or pluripotent progenitor cells and granulocyte-colony stimulating factor (G-CSF), as a combined preparation for simultaneous, separate or sequential use in the therapy of cardiomyopathy in a subject, wherein the cells are administered directly to the myocardium of the subject. Preferably, the G-CSF is administered subcutaneously.

Further according to said first or second aspect, preferably the therapy is treatment, and preferably the subject is diagnosed with ischaemic cardiomyopathy, for example by a qualified clinician. However, cardiomyopathy that is non-ischaemic in origin may also be treated.

Further according to said second aspect, the above cells may be obtainable by bone marrow aspiration. Further according to said first or second aspect, the cells have preferably been obtained by bone marrow aspiration. The procedure may be performed using any suitable method which is available in the art. However, by way of example and not limitation, bone marrow may be obtained from the posterior iliac crest; between 20 and 120 ml, preferably between 30 and 100 ml, more preferably between 40 and 80 ml, most preferably about 50 ml bone marrow may be aspirated from 2, 3, 4 or more different sites over the said iliac crest. The bone marrow aspirate, if obtained correctly, will inherently comprise the desired cells, which will also be retained after correct processing as detailed below. By way of further example and not limitation, peripheral venous blood may also be obtained from the subject (for example, 20-50 ml, preferably 30-40 ml, more preferably about 36 ml), for autologous cells to be suspended therein prior to intramyocardial injection.

Further according to said first or second aspect, said cells may comprise multipotent or pluripotent adult stem cells, preferably CD34+ bone marrow stem cells. Without wishing to be bound by theory, the above cell types may be, but are not necessarily, required for the efficacy of the invention.

Further according to said first or second aspect, in a particularly preferred embodiment the above cells are autologous.

Further according to said first or second aspect, preferably the cells are delivered to the myocardium of the subject by percutaneous injection directly to the mycocardium, for example by using a MyoStar™ injection catheter. More preferably, said percutaneous injection is directed by three-dimensional electromechanical mapping of the subject's heart (preferably the left ventricle). In a particularly preferred embodiment, the above electromechanical mapping and percutaneous injection to the myocardium is performed by a system comprising the instrument sold under the trade name NOGA® XP Cardiac Navigation System as of 10 Jun. 2014, or a suitable variant or derivative instrument thereof, but all suitable 3-D electromechanical mapping systems are included within the scope of the invention. Said instrument simultaneously registers the electrical and mechanical activities of the left ventricle, enabling online assessment of myocardial viability. It is able to distinguish between viable, nonviable, stunned, and hibernating myocardium and can assess wall motion. Using said system, mapping of the left ventricle enables the assessment of endocardial unipolar voltage, for the selection of optimum sites of injection. When using a 3-D electromechanical mapping system comprising, for example, the NOGA® instrument, it is most preferred that regions of the myocardium having a unipolar voltage between 6.9 mV and 11 mV are selected as sites of injection, thereby excluding scarred and overactive tissue through said lower and upper limits, respectively. Any suitable number of injections may be made using the system, into distinct target areas; for example at least 5 injections, preferably at least 6, 7, 8 or 9, most preferably at least 10 injections. Said target areas (or locations) for injection may also be suitably spaced, for example wherein the minimum distance between any two of said locations is between 0.5 and 1.5 cm, preferably between 0.8 cm and 1.2 cm, more preferably about 1 cm, most preferably wherein any two of said locations are no less than 1 cm apart. By way of example and not limitation, about 2 ml total volume of injectate comprising suspended cells may be used over the above numbers of injections. With regard to the wall thickness of the myocardium, as assessed electromechanical mapping (preferably by the above system), preferably areas having a wall thickness of less than 5 mm are avoided.

Further according to said first or second aspect, granulocyte-colony stimulating factor (G-CSF) is administered to the subject, preferably by subcutaneous administration, over a period of at least 2, 3, 4 or 5 days, most preferably 5 days in total. This may be performed using standard methods which are available in the art. Doses may be between 2 and 20 μg/kg/day, preferably between 5 and 15 μg/kg/day, more preferably between 8 and 12 μg/kg/day, most preferably about 10 μg/kg/day. Recombinant human G-CSF is particularly preferred for use in accordance with the present invention (for example, Granocyte®, available from Chugai Pharma as of 10 Jun. 2014; other forms available to the skilled person may also be used).

Further according to said first or second aspect, the cells may be administered within a period of no more than 3, or 2 days, preferably no more than 1 day directly following the end of the above period of G-CSF administration. In one particularly preferred embodiment, bone marrow aspiration is performed within 24 hours prior to administration of the cells derived from the aspirate, even more preferred within 12 hours.

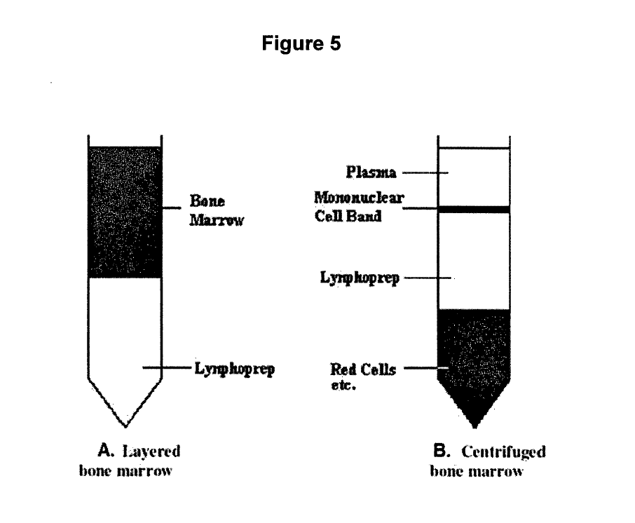

Further according to said first or second aspect, bone marrow aspiration, resulting in a sample obtained from the subject, may be followed by a non-automated (that is, manual) processing method for said sample, and thus the cells for use according to the present invention are preferably obtainable, more preferably have been obtained, by said method. Said method comprises centrifugation of the sample such that mononuclear cells are substantially separated from plasma and non-nucleated cells (and able to be removed using standard techniques, for example aspiration). Any suitable method of centrifugation available to the skilled person may be used which achieves the requisite effect (as exemplified in FIG. 5B). By way of example and not limitation, centrifugation using a suitable density gradient between 1000 and 3000 rpm for between 10 and 50 minutes may be used, preferably between 1500 and 2500 rpm for between 20 and 40 minutes; 1900 rpm for 30 minutes is particularly preferred. Preferably, the centrifugation is performed with the sample in a solution comprising polysaccharide, having sufficient osmolality and density to effect substantial separation of the mononuclear cells, using a centrifugation regime as described above. A suitable example includes ‘Lymphoprep™’ (available from STEMCELL Technologies as of 10 Jun. 2014), having an osmolality of 280±15 mOsm and a density of 1.077±0.001 g/ml. Preferably, the sample is filtered (for example with a filter having a pore size between 50-500 μm, preferably 100-400 μm, more preferably 150-300 μm, most preferably 200 μm) prior to said centrifugation; more preferably, the sample is also washed after filtration and prior to centrifugation (preferably in saline solution, for example 0.1-2%, preferably 0.9%). Following said centrifugation, the sample may be further treated by, sequentially, steps (a)-(d) and, optionally step (e); wherein; step (a) comprises centrifugation of said mononuclear cells to produce a cell pellet (for example between 1500 and 3500 rpm for between 5 and 15 minutes, preferably between 2000 and 3000 rpm for between 8 and 12 minutes, more preferably at 2500 rpm for 10 minutes); step (b) comprises re-suspension of said cell pellet (preferably in saline solution, for example 0.1-2%, preferably 0.9%); step (c) comprises further centrifugation of said re-suspended cell pellet to produce a further cell pellet (for example between 1500 and 3500 rpm for between 5 and 15 minutes, preferably between 2000 and 3000 rpm for between 8 and 12 minutes, more preferably at 2500 rpm for 10 minutes); step (d) comprises re-suspension of said further cell pellet, (preferably in saline solution, for example 0.1-2%, preferably 0.9%); step (e) comprises dilution of the suspension obtained in (d) in autologous serum, obtained from the subject so as to reach the desired volume of injectate (as described above, for example 2 ml). In one specific embodiment, the processing method is performed substantially in accordance, preferably in accordance, with the Standard Operating Procedure described in the Example (below).

According to a further aspect of the invention, there is provided a method of treating and/or preventing cardiomyopathy in a subject in need of prevention or treatment thereof; said method comprising administering a therapeutically effective amount of G-CSF and cells obtainable by bone marrow aspiration, or multipotent or pluripotent progenitor cells, wherein the cells are administered directly to the mycocardium; said method having the same optional and preferred features as are applicable to the first and second aspects of the invention.

According to a further aspect of the invention, there is provided a kit comprising sterile elements (preferably wherein the whole kit is sterile) including cells obtainable by bone marrow aspiration, or multipotent or pluripotent progenitor cells, G-CSF, and instructions for use specifying administration of the cells directly to the myocardium; preferably in addition to an injector configured to deliver the cells directly to the myocardium; preferably further in addition to a system for three-dimensional electromechanical mapping of the heart (preferably the left ventricle).

The invention is now illustrated by reference to the following non-limiting Example. Reference may be made to the Example for further information on how to perform the invention, in conjunction with that provided by the Detailed Description of the Invention.

### Example

REGENERATE-IHD Trial Methodology

The REGENERATE-IHD trial is an investigator-initiated, single-centre, randomised placebo-controlled trial. The trial assessed G-CSF administration alone and in combination with either the intracoronary or intramyocardial injection of autologous bone marrow derived cells (BMC) versus matching placebo controls (serum). Following a pilot study to address safety and feasibility the Local Research Ethics Committee approved the protocol (REC no. 04/Q0603/13) and the trial was conducted in accordance with the Declaration of Helsinki. The trial was registered with clinicaltrials.gov (NCT00747708) and the European Clinical Trials register (EudraCT no. 2005-002706-27). Written informed consent was obtained from each patient prior to inclusion in the trial with all adverse events reported to an independent safety monitoring board.

Patients were recruited to the trial with a confirmed diagnosis of heart failure from local heart failure clinics. Patients fulfilling all of the following criteria were included: confirmed diagnosis of heart failure secondary to ischaemic heart disease, established on maximal medical therapy for at least 6 months, documented impaired left ventricular systolic function, NYHA II-IV and no further treatment options. Exclusion criteria included acute coronary syndrome within the preceding 6 months, cardiogenic shock, atrial fibrillation, impaired renal function (serum creatinine >200 μmol/l), serious concomitant illness with a life expectancy of <1 year, contraindication to bone marrow aspiration, chronic inflammatory disease, active infection, known infection or high-risk lifestyle for infection with human immunodeficiency virus, hepatitis B virus, hepatitis C virus, syphilis or HTLV (Human T-cell lymphotropic virus).

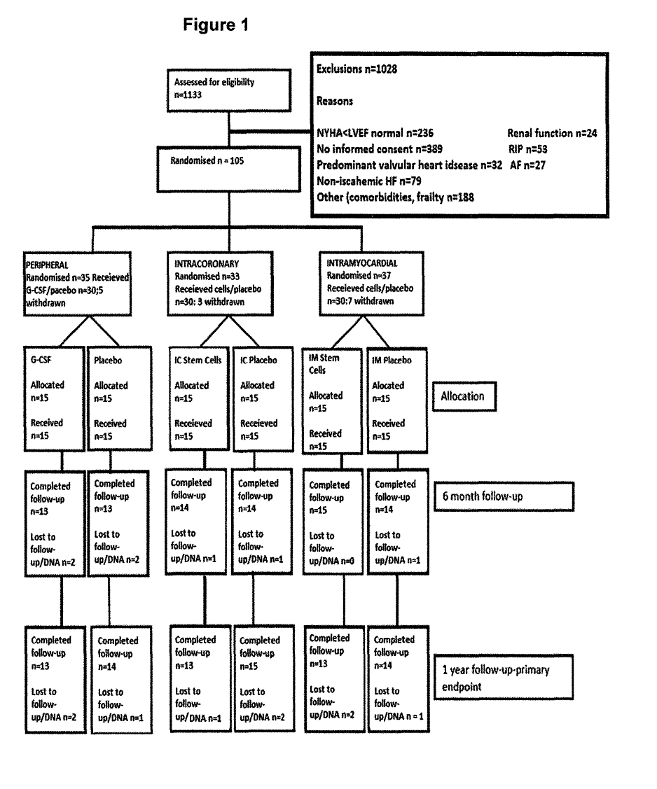

After consenting for the trial, patients were randomized to 1 of 3 arms using a dedicated trial software system (IHD Clinical, Bishops Stortford, Herts, UK) (1:1:1—simple randomisation algorithm). In the peripheral arm patients received either saline or subcutaneous G-CSF (10 μg/kg/d). In the interventional arms patients received 5 days of subcutaneous G-CSF (10 μg/kg/d) prior to bone marrow harvest and same day injection of cells or serum either by the intracoronary or intramyocardial route (NOGA® system) (FIG. 1).

Recombinant human G-CSF (Granocyte®, Chugai Pharma, UK) was administered subcutaneously at a dose of 10 μg/kg/day for 5 consecutive days prior to bone marrow harvest on Day 6. Patients had routine blood tests performed daily as well as a sample taken for CD34+ cell count estimation.

Bone marrow was obtained from the posterior iliac crest. 50 ml of bone marrow were aspirated equally into heparin-treated syringes from 3 separate sites over the iliac crest. 36 ml of peripheral venous blood was acquired immediately prior to bone marrow harvest to obtain autologous serum for intramyocardial injections. Blood and bone marrow samples were delivered immediately to the Good Clinical Practice accredited Stem Cell Laboratory for processing. Isolation and characterisation of BMSCs were performed by a designated lab technician.

The bone marrow sample was processed manually in accordance with the Standard Operating Procedure given below. Briefly, the bone marrow sample was layered on a density gradient medium (Axis shield, Oslo, Norway) and centrifuged at 2500 rpm for 30 minutes. The mononuclear cell fraction was extracted and subjected to 3 wash cycles in 0.9% saline (Baxter, Norfolk, UK). More details are provided in the Standard Operating Procedure, below. Cells were resuspended in 2 ml of autologous serum for intramyocardial injection.

Control group injections consisted of 2 ml autologous serum alone. Samples were maintained at room temperature for the entire procedure and the final injectate of stem/progenitor cell suspension or placebo was transported to the cardiac catheter laboratory at London Chest Hospital for the intramyocardial or intracoronary injection procedure. Viability of the cell preparation was checked with 7-AAD (7-aminoactinomycin D) staining immediately prior to infusion and was 98.4±0.7% in the cell treated group.

Bone marrow and peripheral blood circulating progenitor cells (CD34+ cells and endothelial progenitor cells (EPCs)) were characterised using flow cytometry. All flow cytometry analyses were performed using a BD FACSCanto Flow Cytometer with BD FACSDiva v 5.0.3 software (BD Biosciences). For the identification of HSC populations, cells were incubated with fluorescein isothiocyanate (FITC)-labelled antibody against human CD45 (BD Biosciences, Erembodegem-AALST, Belgium) and phycoerythrin (PE)-103 labeled antibody against human CD34 (BD Biosciences) for 15 min at room temperature.

EPCs were analysed by initially incubating samples with mouse serum IgG (Sigma, Dorset, UK) for 15 min at 4° C. with a cocktail of antibodies comprising allophycocyanin (APC)-labelled antibody to CD133 (Miltenyi Biotec, Surrey, UK) and PE-labelled antibody to VEGFR-2 (R&D Systems, Abingdon, UK) to characterise EPCs and FITC-labelled monoclonal antibodies to CD2, CD13 and CD22 (Beckman Coulter, High Wycombe, UK) to identify and therefore eliminate inclusion of lineage-negative non-progenitor cells. To ensure exclusion of nonviable cells in the final EPC count, cells were also incubated with a PerCP-Cy5-labelled 7AAD stain (BD Biosciences). Cells were then incubated for 15 min at room temperature with 2 ml of Pharm Lyse™ buffer (BD Biosciences) to lyse red blood cells. Samples were washed once in phosphate-buffered saline and 20 μl of Accucount flow cytometry beads (Saxon Europe, Kelso, UK) were added before analysis.

Functional analysis of CD34+ cells was performed using a colony-forming unit (CFU-GM) assay. BM-MNCs (2×10⁴ per dish), Day 0 peripheral blood mononuclear cells (MNCs) (2×10⁵ per dish), and Day 6 peripheral blood MNCs (2×10⁴ per dish) were seeded, in triplicate preparations, in methylcellulose plates (Methocult H4534, including stem cell factor, granulocyte-macrophage colony-stimulating, and interleukin-3, Stem cell Technologies). Plates were studied under phase contrast microscopy, and granulocyte-macrophage colony forming units (CFU GM; colonies >50 cells) were counted after 14 days of incubation. Results were taken from the mean of the triplicate results.

Intramyocardial injection was performed using the NOGA® mapping system and MyoStar™ injection catheter. After femoral arterial access (8Fr sheath), a weight adjusted bolus dose of heparin was given as per routine procedure. A LV angiogram was initially performed to assess LVEF and also to guide intramyocardial injection. Patients then underwent LV electromechanical mapping using NOGA® XP Cardiac Navigation System (Biologics Delivery Systems Group, Cordis Corporation, Diamond Bar, Calif., USA) to delineate the scar area (unipolar voltage <6.9 mV) and surrounding viable but hibernating myocardium; areas of myocardium with a unipolar voltage of greater than 11 mV were avoided (overactive myocardium). This was correlated with areas of wall dyskinesia on the LV angiogram. Direct intramyocardial injection was performed with the MyoStar™ injection catheter (Biologics Delivery Systems Group, Cordis Corporation, Diamond Bar, Calif., USA) as previously described (Perin et al., 2004; Perin et al., 2002). The total 2 ml volume of injectate was delivered equally over 10 target areas at approximately 1 cm intervals. Areas of the myocardium with a wall thickness of <5 mm were avoided. The electromechanical mapping data (unipolar voltages and local linear shortening) were recorded so that they could be compared to the follow-up mapping procedure performed at 6-months follow-up.

The primary end-point for all patients was the change in left ventricular ejection fraction (LVEF) at 12 months compared to baseline as measured using advanced cardiac imaging (cardiac magnetic resonance imaging (CMR) or computed tomography (CT) where CMR contraindicated). Secondary endpoints included change in NT-pro BNP, NYHA, quality of life (assessed by EQ5D®, SF-36® and MacNew® questionnaires) and LVEF assessed using contrast transthoracic echocardiogram (TTE) and Quantitative Left Ventriculography (QLV) (in the IC and IM groups only) at 6 months compared to baseline. Additional secondary endpoints at 1 year were change in NYHA class, LVEF measured by contrast TTE and quality of life (QoL) compared to baseline. Major adverse cardiac events (MACE; defined as cardiac death, myocardial infarction (MI), percutaneous coronary intervention (PCI) or CABG) or any significant arrhythmias (defined as symptomatic ventricular tachycardia or survived cardiac death) were assessed at 6 months and 1 year.

CMR or cardiac CT for those unable to undergo CMR (e.g. cardiac devices, claustrophobia etc.) were performed at baseline and 12 months. Multi-phase cardiac data sets with full left ventricular coverage were acquired using standard protocols.

Following a pilot study, as requested by the ethics committee, the study was powered to detect a 3.5 percentage point increase within group improvement in the primary endpoint i.e. change in LVEF at 12 months based on changes seen in a contemporary trial of cell therapy. Based on a power of 90%, a significance level of 5% and an estimated within observation error of 4%, the calculated required number of patients in each group was 11. It was estimated that an additional 4 per group would be needed in order to ensure that 11 patients reached the primary endpoint at 1 year, resulting in a size of 15 patients per treatment group.

A paired t-test was used to detect any statistical significance of within group changes in LVEF. For additional analyses using continuous variables, appropriate parametric (paired-t for paired and independent samples t-test for non-paired data, one-way ANOVA for multiple comparisons) and non-parametric (Wilcoxon signed-rank test for paired and Mann-Whitney for non-paired data) tests were used. Chi-squared or Fisher's exact tests were used for categorical variables. Pearson's linear regression was used for comparison between LVEF and cell function variables. Values are quoted as mean±SD unless otherwise stated. All p-values were two sided and p<0.05 was accepted to denote statistically significance. Statistical analyses were performed using SPSS® version 21 (IBM) and graphs produced using Graphpad Prism® version 5.0 (GraphPad Software, San Diego, Calif.). The analyses were reviewed by the trial statistician.

TABLE 1
Patient Baseline Characteristics

| | Saline | G-CSF | IC serum | IC BMC | IM serum | IC BMC | p- |
|---|---|---|---|---|---|---|---|
| | (n = 15) | (n = 15) | (n = 15) | (n = 15) | (n = 15) | (n = 15) | value |
| Age, years | 63.3 ± 9.3 | 63.1 ± 8.2 | 62.3 ± 11.0 | 62.1 ± 9.7 | 60.4 ± 11.2 | 65.3 ± 9.4 | 0.888 |
| (mean ± SD) | | | | | | | |
| Sex M/F n | 15/0 | 13/2 | 14/1 | 14/1 | 15/0 | 15/0 | 0.388 |
| BMI (kg/m²) | 29.5 ± 4.3 | 31.4 ± 6.0 | 31.7 ± 6.5 | 29.7 ± 4.8 | 29.6 ± 3.7 | 30.8 ± 4.0 | 0.789 |
| (mean ± SD)* | | | | | | | |
| Medical History, n (%) | | | | | | | |
| Hypertension | 4 (26.7) | 1 (6.7) | 2 (13.3) | 4 (26.7) | 5 (33.3) | 5 (33.3) | 0.412 |
| Diabetes | 4 (26.7) | 5 (33.3) | 3 (20.0) | 2 (13.8) | 4 (26.7) | 4 (26.7) | 0.859 |
| CABG | 5 (33.3) | 4 (26.7) | 3 (20.0) | 7 (46.7) | 6 (40.0) | 4 (26.7) | 0.653 |
| MI | 18 (86.7) | 12 (80.0) | 14 (93.3) | 13 (88.7) | 13 (88.7) | 13 (86.7) | 0.949 |
| Hypercholesterolaemia | 6 (40.0) | 6 (40.0) | 5 (33.3) | 5 (33.3) | 8 (53.3) | 4 (33.3) | 0.754 |
| Smoker/ex-smoker, | 12 (80.0) | 8 (58.8) | 13 (86.7) | 11 (73.3) | 14 (98.3) | 11 (73.8) | 0.150 |
| Time from fast MI, | 1307 (1064-5443) | 2527 (866-4928) | 2856 (1278-6041) | 1805 (995-3855) | 2406 (786-5402) | 2884 (786-5402) | 0.964 |
| days median (IQR) | | | | | | | |
| LVEF (%) | 34.7 ± 10.1 | 27.8 ± 12.4 | 30.6 ± 8.1 | 32.6 ± 8.1 | 29.0 ± 9.2 | 28.8 ± 18.2 | 0.385 |
| (mean ± SD) | | | | | | | |
| Devices number, n (%) | | | | | | | |
| CRT-D | 4 (26.7) | 5 (33.3) | 4 (28.7) | 4 (26.7) | 3 (20.0) | 7 (46.7) | 0.781 |
| CRT-P | 1 (8.7) | 1 (6.7) | 0 (0) | 0 (0) | 0 (0) | 0 (0) | 0.999 |
| ICD only | 7 (46.7) | 2 (13.3) | 4 (26.7) | 5 (33.3) | 5 (33.3) | 6 (40.0) | 0.485 |
| Medication history, | | | | | | | |
| n (%) | | | | | | | |
| Statin | 12 (80) | 13 (85.7) | 13 (86.7) | 12 (80) | 14 93.3) | 13 (86.7) | 0.910 |
| ACEI/ARB | 14 (93.3) | 14 (93.3) | 13 (86.7) | 13 (86.7) | 15 (100) | 15 (100) | 0.509 |
| B-blocker | 14 (93.3) | 14 (93.3) | 11 (73.3) | 14 (93.3) | 11 (73.3) | 14 (93.8) | 0.226 |
| Aldosterone antagonist | 8 (58.3) | 13 (86.7) | 9 (60.0) | 12 (80.0) | 9 (60.0) | 12 (80.0) | 0.284 |
| Diuretics | 10 (66.7) | 11 (73.3) | 13 (86.7) | 12 (80.0) | 8 (53.3) | 12 (80.0) | 0.363 |
| NYHA at baseline, | | | | | | | |
| n (%) | | | | | | | |
| II | 10 (66.7) | 9 (60.0) | 5 (88.3) | 8 (53.8) | 11 (73.3) | 8 (58.3) | 0.376 |
| III/IV | 5 (33.8) | 6 (40.0) | 10 (66.7) | 7 (46.7) | 4 (26.7) | 7 (45.7) | |

### REGENERATE-IHD Trial Results

A total of 1133 patients were referred from heart failure clinics throughout the UK. One thousand and twenty eight were ineligible due to the following reasons: non-ischaemic aetiology of heart failure (n=79), NYHA<II/normal LVEF (n=236), atrial fibrillation (n=27), refusal to participate in the trial (n=389), death before formal consent (n=53) and other comorbidities, including valvular heart disease and impaired renal function (n=244). Of the 105 patients who were randomised, 90 received the allocated trial intervention (FIG. 1).

The mean age of the patient population was 62.86±9.7 years while the majority of patients were male (95.6%). The baseline characteristics were similar across the groups with a mean LVEF of 30.6±1.1%, a mean NT-pro-BNP of 898.9±131.4 pg/ml and 97.8% of patients in NYHA class Baseline patient demographics are shown in Table 1. The majority of patients were on optimal medical therapy (as per ESC guidelines), and there were no significant differences across groups in prescribed medication or implanted device therapy.

A total of 82 patients were assessed for the primary endpoint at 1 year. At 1 year in the IM arm, 14 patients in the BMC group were assessed (1 referred for LVAD) with 13 patients assessed (1 death and 1 patient lost to follow-up) in the IM placebo group. In the IC arm, all 15 patients in the BMC group and 13 patients in the placebo group (1 death and 1 CT scan not analysable) were assessed for the primary end-point. In the peripheral group, 13 patients in the G-CSF group (1 death and 1 CT scan not analysable) and 14 in the placebo group (1 patient lost to follow-up) were assessed for the primary endpoint.

Only patients treated in the IM BMC group met the primary endpoint of change in LVEF (increase of 4.99 percentage point, 95% CI 0.33-9.6%; p=0.038). In post hoc analysis compared to the peripheral placebo arm an absolute change in LVEF of 5.97 percentage points (p=0.035) was seen. Although a trend to improvement in LVEF was seen in the IM serum group {4.15 percentage points (95% CI −3.3-11.6%)}, this did not reach significance. Importantly no change was seen in the IC cell treated group (0.66 percentage points; 95% Cl −2.4-3.7%; p=0.649). In the peripheral arm, there was a decrease in LVEF in both groups: GCSF (−1.25 percentage points; 95% Cl −5.4-2.9%; p=0.520) and placebo (−0.98 percentage points; 95% Cl −4.4-2.5%; p=0.551). The improvements in cardiac function in the IM BMC group was also seen using echocardiography (FIG. S2, supplemental data). There were no significant changes in LV end-diastolic or end-systolic volumes (LVEDV, LVESV) over time in any treatment group.

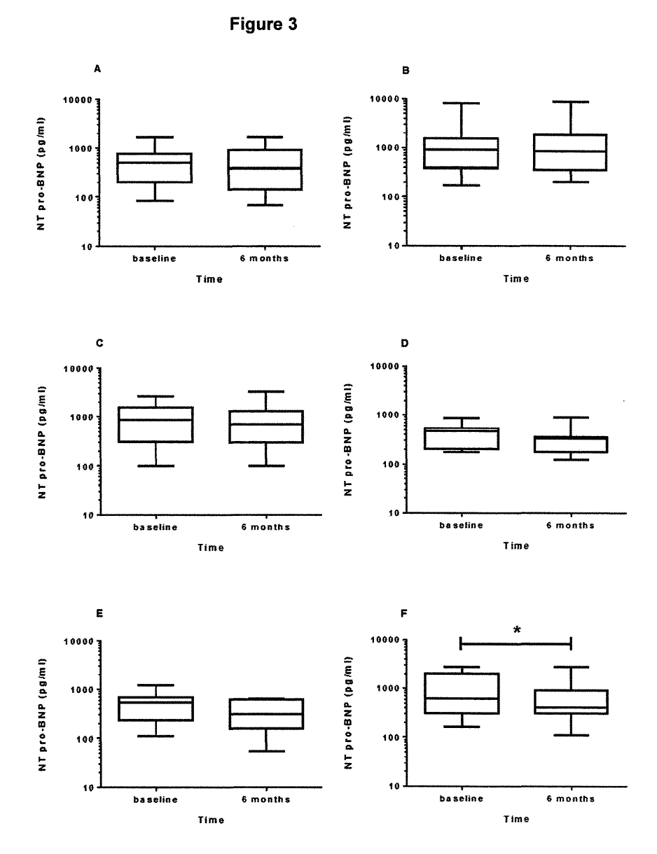

Blood analysis was performed on samples at baseline, and 6 months for each patient; statistical analysis was performed after logarithmic transformation of all NT-proBNP values due to a non-normal distribution. At 6 months, only patients in the IM BMC group showed a significant fall in NT pro-BNP (977.5±866.8 to 768.4±754.4; p=0.018). The remaining groups showed no significant changes in NT pro-BNP levels at 6 months. (FIG. 3)

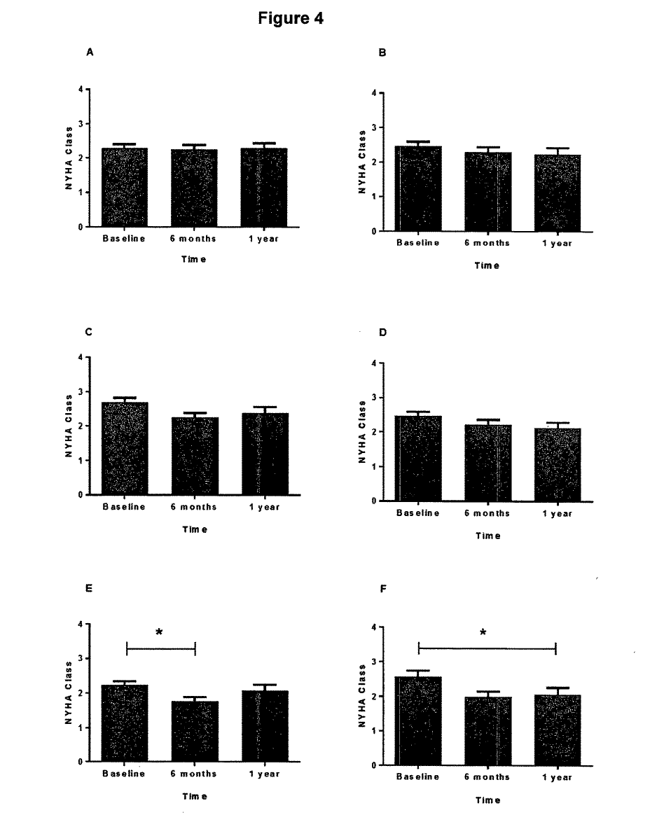

In the IM BMC group, there was a significant improvement in NYHA class at 6 months (2.57 to 2.00; p=0.025), which was maintained at 1 year (2.57 to 2.07; p=0.047). In the IM serum group, a significant reduction in NYHA was seen at 6 months (2.23 to 1.77; p=0.022) but this was not sustained at 1 year (2.23 to 2.08; p=0.420). In all other groups, no significant change in NYHA class from baseline to 6 months or baseline to 1 year was seen (FIG. 4). Baseline CCS class was similar across all groups and there was no significant change in CCS class at 6 months or 1 year in any group.

The IM BMC group showed an improvement in QoL as assessed by the physical wellbeing score in the SF36® questionnaire at 1 year with no significant improvement seen in other questionnaires. In comparison, the IM placebo group showed no significant improvement in any questionnaire at 1 year. In the IC BMC group there was a significant improvement in both the MacNew® and EQ5D VAS at 1 year, in comparison the IC placebo group showed an improvement in the SF36® physical wellbeing score at 1 year however this was associated with a conflicting significant decline in the SF36® mental wellbeing score. Neither group in the peripheral arm showed any significant improvement in QoL at 1 year.

The total number of cells injected in the two cell groups ranged from 15.3×10⁶ to 296.1×10⁶ BMMNC. The mean viability of processed cells was 98.2%. A significant correlation was observed between the number of CFU-GM colonies and the improvement in LVEF in the IM BMC group (Pearson r=0.79; p=0.02). No significant correlation was observed between bone marrow CFU-GM and change in LVEF in the IC BMC group.

A small increase in post-procedural troponin was detected in the IM arm (0.02±0.04; p=0.007), however analysis by treatment group, revealed no significant troponin increase in the IM BMC group (0.01±0.04, p=0.228) with the increase restricted to the IM placebo group (0.03±0.04; p=0.013). Clinically this rise is insignificant and was not associated with a significant rise in creatine kinase (85.1 to 129.5; p=0.102). Importantly no clinical events occurred during the procedures. No significant increases in cardiac enzymes were seen in any other treatment arm. No significant differences in procedural complication rates (p=0.363), significant arrhythmias (p>0.999) or MACE rates (p>0.999) were noted between the groups. MACE rates were low for all groups (one cardiovascular death in G-CSF group and one MI in the IC placebo group).

### Bone Marrow Processing Standard Operating Procedure

1. The Bone Marrow is passed through into the Cell Culture Laboratory.
2. The harvest is passed through a 200 μm filter and pooled into one bag; the filter line is flushed with 0.9% saline.
3. Using a sterile needle and 5 mL syringe take off 3 ml of the bone marrow harvest and ensure that the harvest is well mixed (spray the injection site with 70% sterile filtered ethanol).
4. Place 1 ml of the harvest in one 2 ml labelled cryovial for the procedure pre counts, 1 ml each in the labelled of ‘Bact Alert’ culture bottles.
5. Swab the appropriate number of tube racks, spray with the ethanol and place in laminar flow cabinet.
6. In the laminar flow cabinet label the appropriate amount of 50 ml conical tubes, noting that each tube will take 25 mls of bone marrow.
7. Carefully remove the screw caps of the 50 ml tubes placing them upside down in the cabinet and add 25 mls of ‘Lymphoprep’ (polysaccharide solution of osmolality and density (280±15 mOsm, 1.077±0.001 g/ml) using a syringe and filling tube.
8. Take up bone marrow into 50 ml, add a filling tube and carefully layer the bone marrow onto the ‘Lymphoprep’ ensuring that no mixing occurs (see FIG. 5a).
9. Once all the bone marrow is layered onto the ‘Lymphoprep’ tightly screw the lids back on.
10. Using Centrifuge in Cell Processing Lab, centrifuge the tubes at 1900 rpm for 30 mins with the brake set to ‘1’. No Brake.
11. Carefully remove the tubes from the centrifuge and place back in the laminar flow cabinet. After centrifugation the mononuclear cells form a distinct band (see FIG. 5b). Label between 4 and 8 more 50 ml tubes.
12. In the laminar flow cabinet use a sterile pastette to aspirate the distinct band of mononuclear cells and transfer to an empty 50 ml tube. Repeat until all of the band has been aspirated and then discard the waste. It is better to take too much from either side of the band than too take too little and to risk losing cells; see FIG. 5.
13. Repeat this for all the tubes until all the cell bands have been transferred to new tubes.
14. Centrifuge these tubes at 2500 rpm for 10 minutes. Return the tubes to the laminar flow cabinet and carefully tip off the supernatant in to a sterile 50 ml tube.
15. To each of the cell pellets add 3-5 ml of 0.9% Saline re-suspend using a sterile pastette.
16. Transfer the cell suspensions to one tube and repeat washing the tubes a further two times.
17. Discard the empty tubes once completed.
18. Fill the remaining tube to the 50 ml level and centrifuge at 2500 rpm for 10 minutes. Remove and discard the supernatant in the laminar flow cabinet.
19. Re-suspend the cell pellet to in 0.9% saline to 13 mls or 5 mls depending on randomisation group.
20. Add 1 ml of the cell suspension to each of the ‘Bact Alert’ bottles and 1 ml into cryovial for analysis.
21. Take up required volume into the syringe and wrap with label so as to obscure contents. Label with patient details: Trial No, date of birth, unique recogniser number, date and volume.
22. Place tamper proof syringe cap onto syringe and put into sterile paper bag provided. Place into overwrap bag and heat seal. Pass out through Preparation area for immediate transportation in cool bag.
23. Record Lot numbers used and Analysis Results on Cardiac Trial Worksheet.

### REFERENCES

Assmus B, Fischer-Rasokat U, Honold J, et al. Transcoronary transplantation of functionally competent BMCs is associated with a decrease in natriuretic peptide serum levels and improved survival of patients with chronic postinfarction heart failure: results of the TOPCARE-CHD Registry. Circulation research. Apr. 27 2007, 100(8):1234-1241.
Baldazzi F, Jorgensen E, Ripa R S, Kastrup J. Release of biomarkers of myocardial damage after direct intramyocardial injection of genes and stem cells via the percutaneous transluminal route. European heart journal. August 2008; 29(15):1819-1826.
Chih S, Macdonald P S, McCrohon J A, et al. Granulocyte colony stimulating factor in chronic angina to stimulate neovascularisation: a placebo controlled crossover trial. Heart. February 2012; 98(4):282-290.
Hill J M, Syed M A, Arai A E, et al. Outcomes and risks of granulocyte colony-stimulating factor in patients with coronary artery disease. Journal of the American College of Cardiology. Nov. 1 2005; 46(9):1643-1648.
Honold J, Fischer-Rasokat U, Lehmann R, et al. G-CSF stimulation and coronary reinfusion of mobilized circulating mononuclear proangiogenic cells in patients with chronic ischemic heart disease:five-year results of the TOPCARE-G-CSF trial. Cell transplantation. 2012; 21(11):2325-2337.
Joseph J, Rimawi A, Mehta P, et al. Safety and effectiveness of granulocyte-colony stimulating factor in mobilizing stem cells and improving cytokine profile in advanced chronic heart failure. The American journal of cardiology. March 1 2006; 97(5):681-684.
Jeevanantham V, Butler M, Saad A, Abdel-Latif A, Zuba-Surma E K, Dawn B. Adult bone marrow cell therapy improves survival and induces long-term improvement in cardiac parameters: a systematic review and meta-analysis. Circulation. Jul. 31 2012; 126(5):551-56
MacIntyre K, Capewell S, Stewart S, et al. Evidence of improving prognosis in heart failure: trends in case fatality in 66 547 patients hospitalized between 1986 and 1995. Circulation. Sep. 5 2000, 102(10):1126-1131.
Mosterd A, Cost B, Hoes A W, et al. The prognosis of heart failure in the general population: The Rotterdam Study. European heart journal. August 2001; 22(15):1318-1327.
Perin E C, Dohmann H F, Borojevic R, et al. Transendocardial, autologous bone marrow cell transplantation for severe, chronic ischemic heart failure. Circulation. May 13 2003; 107(18):2294-2302.
Wang Y, Tagil K, Ripa R S, et al. Effect of mobilization of bone marrow stem cells by granulocyte colony stimulating factor on clinical symptoms, left ventricular perfusion and function in patients with severe chronic ischemic heart disease. International journal of cardiology. Apr. 28 2005; 100(3):477-483.
Yeo C, Mathur A. Autologous bone marrow-derived stem cells for ischemic heart failure: REGENERATE-IHD trial. Regenerative medicine. January 2009; 4(1):119-127.

1. A composition comprising:
a) cells obtainable by bone marrow aspiration and granulocyte-colony stimulating factor (G-CSF), as a combined preparation; or
b) multipotent or pluripotent progenitor cells and granulocyte-colony stimulating factor (G-CSF), as a combined preparation,
wherein said composition is formulated as a combined preparation for simultaneous, separate or sequential use in the therapy of cardiomyopathy in a subject, such that the cells are administered directly to the myocardium of the subject.

2. (canceled)

3. The composition according to claim 1, wherein said cells have been obtained by bone marrow aspiration.

4-5. (canceled)

6. The composition according to claim 1, wherein said cells comprise CD34+ bone marrow stem cells.

7. The composition for use according to claim 1, wherein said cells are autologous.

8-16. (canceled)

17. The composition according to claim 3, wherein said cells are obtainable by a non-automated method comprising; after the bone marrow aspiration, centrifugation of the sample obtained from the subject by said bone marrow aspiration, performed such that mononuclear cells are substantially separated from plasma and non-nucleated cells.

18. The composition according to claim 17, wherein the centrifugation is performed on the sample when in a solution comprising polysaccharide, the solution having sufficient osmolality and density to substantially separate mononuclear cells from plasma and non-nucleated cells.

19. The composition according to claim 17, wherein said method comprises filtration, and washing of the sample with saline solution, prior to the centrifugation thereof.

20. The composition according to claim 17, wherein said method further comprises the steps (a)-(d) and, optionally step (e);
(a) centrifugation of said mononuclear cells to produce a cell pellet,
(b) re-suspension of said cell pellet, preferably in saline solution,
(c) further centrifugation of said re-suspended cell pellet to produce a further cell pellet,
(d) re-suspension of said further cell pellet, preferably in saline solution, and, optionally,
(e) dilution of the suspension obtained in (d) in autologous serum, obtained from the subject.

21. A kit comprising sterile elements including cells obtainable by bone marrow aspiration, or multipotent or pluripotent progenitor cells, G-CSF, and at least one of:
a) instructions for use specifying administration of the cells directly to the myocardium; and
b) an injector configured to deliver the cells directly to the myocardium.

22. (canceled)

23. The kit comprising sterile elements according to claim 22, further comprising a system for three-dimensional electromechanical mapping of the heart.

24. A method of treating or preventing cardiomyopathy in a subject in need of such treatment or prevention; said method comprising administering to the subject a therapeutically effective amount of:
a) cells obtainable by bone marrow aspiration and granulocyte-colony stimulating factor (G-CSF), and/or
b) multipotent or pluripotent progenitor cells and granulocyte-colony stimulating factor (G-CSF),
wherein said cells and G-CSF are administered simultaneously, separately or sequentially and, wherein the cells are administered directly to the myocardium of the subject.

25-26. (canceled)

27. The method according to claim 24, wherein the cardiomyopathy is ischaemic cardiomyopathy.

28. The method according to claim 24, wherein the cardiomyopathy is non-ischaemic in origin.

29. The method according to claim 24, wherein said cells are administered by percutaneous intramyocardial injection.

30. The method according to claim 24, wherein prior to administration of the cells, three-dimensional electromechanical mapping of the subject's heart is carried out, and the results of the mapping used to determine a site or sites of administration.

31. The method according to claim 30, wherein the cells are administered directly to at least one region of the myocardium having a unipolar voltage of between 6.9 mV and 11 mV, as determined by said electromechanical mapping.

32. The method according to claim 30, wherein the cells are administered directly to at least one region of the myocardium having a myocardial wall thickness of at least 5 mm, as determined by said electromechanical mapping.

33. The method according to claim 24, wherein the cells are administered to at least 5 different locations within the myocardium, the minimum distance between any two of said locations being about 1 cm.

34. The method according to claim 24, wherein the G-CSF is administered subcutaneously.

35. The method according to claim 24, wherein the G-CSF is administered over a period of at least 5 days.

36. The method according to claim 24, wherein the cells are administered within one day directly following the end of G-CSF administration.
